(19) United States
(12) Patent Application Publication
(10) Pub. No.: US 2013/0237712 A1
(43) Pub. Date: Sep. 12, 2013

(54) METHOD FOR ISOLATING FLAVONOID DIHYDROQUERCETIN (TAXIFOLIN) FROM CONIFER WOOD SPECIES

(71) Applicants: Sergey V. Philippov, Moscow (RU); Igor M. Bogorodov, Moscow (RU)

(72) Inventors: Sergey V. Philippov, Moscow (RU); Igor M. Bogorodov, Moscow (RU)

(73) Assignee: FLAVITPURE, INC., Cheyenne, WY (US)

(21) Appl. No.: 13/417,656

(22) Filed: Mar. 12, 2012

(51) Int. Cl. C07D 311/40

(52) U.S. Cl. 549/400

(57) ABSTRACT

A method for isolating flavonoid Dihydroquercetin (taxifolin) from wood parts including lower portions of the trunk, referred to as butt logs, bark and roots. The method includes extracting either the particle-reduced chip fraction obtained by chipping or subdividing into chips the butt logs and roots, or dry cork fraction of the bark or powdered bark obtained as a residue in finishing of mechanical wood products with a polar solvent. The method further includes recovering the extract. The wood used in the method is a hardwood or a coniferous wood selected from wood of Larix genus, spruce wood of the genus Picea, fir wood of the genus Abies, pine wood of the genus Pinus, and wood of Pseudotsuga genus. The isolated prime substance from all of the above genus is a flavonoid such as Dihydroquercetin (taxifolin).

### REFERENCES

[1]. Tatjana Stevanovic, Papa Niokhor Diouf and Martha Estrella Garcia-Perez (2009). Bioactive Polyphenols from Healthy Diets and Forest Biomass. Current Nutrition & Food Science, Vol. 5, No. 4, p. 264-295.

[1a] Lee S B, Cha K H, Selenge D, Solongo A, Nho C W (2007). The chemopreventive effect of taxifolin is exerted through ARE-dependent gene regulation. Biol Pharm Bull., 30(6): 1074-1079.

[1b] Gupta M B, Bhalla T N, Gupta G P, Mitra C R, Bhargava K P. (1971). Anti-inflammatory activity of taxifolin. Japan J Pharmacol., 21(3):377-82.

[2]. Schijlen E G W, de Vos C H R, van Tunen A J, Bovy A G. (2004) Modification of flavonoid biosynthesis in crop plants. Phytochemistry. 65: 2631-48.

[3]. Pew, John C., 1947. A flavanone from Douglas-fir heartwood. J. Am. Chem. Soc., 70 (9), pp 3031-3034.

[4]. E. F. Kurth. Harry J. Kiefer, and James K. Hubbard, (1948). Utilization of Douglas-fir Bark The Timberman, Vol. 49, No. 8, pp. 130-1.

[5]. H. M. Graham, E. F. Kurth, (1949). Constituents of Extractives from Douglas Fir. Ind. Eng. Chem., 41 (2), pp 409-414.

[6]. Migita, Nobuhiko,-Nakano, Junzs, Sakai, Isamu, and Ishi, Shoichi, (1952). Japan Tech. Assoc. Pulp Paper Ind. 6:476-480.

[7]. Kurth, E. F., and Chan, F. L., (1953). “Extraction of Tannin and Dihydroquercetin from Douglas Fir Bark.” J. Amer. Leather Chem. Assoc. 48(1):20-32. Abstr. Bull. Inst. Pap. Chem. 23:469.

[8]. G. M. Barton, J. A. F. Gardner. (1958). Determination of Dihydroquercetin in Douglas Fir and Western Larch Wood. Anal. Chem., 30 (2), pp 279-281.

[9]. G. V. Nair and E von Rudloff. (1959). THE CHEMICAL COMPOSITION OF THE HEARTWOOD EXTRACTIVES OF TAMARACK (LARIX LARICINA (DU ROI) K. K0CH)1. Can. J. Chem., Vol. 37. pp.1608-1613.

[10]. Tyukavkina, N. A., Lapteva, K. I., Larina V. A., (1967). Extractives of Larix dahurica. Quantitative content of quercetin and dihydroquercetin. Chemistry of Natural Substances. Issue 5, pages 298-301.

[11]. Pietarinen S P, Willfor S M, Vikstrom F A. Holmbom B R. (2006) Aspen knots, a rich source of flavonoids. J Wood Chem Technol., 26: 245-58.

[12]. Conde E. Cadahia E, Garciavallejo M. Tomasbarberan F. (1995) Lowmolecular-weight polyphenols in wood and bark of Eucalyptus globulus. Wood Fiber Sci., 27: 379-83.

[13]. Antonova, G. F. 1980. Zapasi, sostav i svojstva drevesini listvennitzej. In “Issledovaniya v oblasti drevesiny i drevesnykh materialov”. Institut lesa i drevesiny, Krasnoyarsk, 6-18.

[14]. G. V., Nair and E. von Rodloff. (1959). THE CHEMICAL COMPOSITION OF THE HEARTWOOD EXTRACTIVES OF TAMARACK (LARIX LARICINA (DU ROI) K. K0CH). CANADIAN JOURNAL OF CHEMISTRY. VOL. 37. Issued as N.R.C. No. 5295.

[15]. HENRIK OUTTRUP, KJELD SCHAUMBURG and JORGEN OGAARD MADSEN. (1985). ISOLATION OF DIHYDROMYRICETIN AND DIHYDROQUERCETIN FROM BARK OF PINUS CONTORTA. Carlsberg Res. Commun. Vol. 50, p. 369-379

### FIELD OF THE INVENTION

This invention relates to a method for isolating wood extractives, i.e. flavonoid Dihydroquercetin (taxifolin), from conifer wood species comprising butt logs, bark and roots.

### BACKGROUND OF THE INVENTION

It is known that the forest biomass is the most important biomass on Earth, and as wood industry is generating the huge amounts of residues, which are available as an important vegetable resource for further processing and valorization of bioactive flavonoid molecules through extraction, which are more or less hydrophilic. The extractable flavonoids, obtainable by solvent extraction of the forest biomass, are of special interest as they are readily available from different types of forest and wood transformation residues. The foods rich in these flavonoids are reviewed along with the forest sources of the same classes of molecules. The emphasis is put on residues of wood transformation such as bark, butt logs roots and knotwood as these materials represent particularly rich resources for flavonoids, particularly Dihydroquercetin (taxifolin). One of the most notorious bioactive properties of flavonoids is their antioxidant activity. The most important results on antioxidant capacity of forest trees extracts are presented and compared to those obtained for the extracts from healthy foods rich in bioactive flavonoid molecules. [1] Flavonoid Dihydroquercetin (taxifolin) is one of the most effective natural antioxidants and anti-inflammatory compound. [1a,b]

The flavonoids are the derivatives of phenylpropanoid metabolism. Their structures are based on C6-C3-C6 skeletons, the A ring of the flavonoid structure being acetate derived (3×C2) and the C and B rings originating from cinnamic acid derivatives (phenylpropanoid pathway). The flavonoids constitute an enormous class of natural polyphenols with more than 6000 [2] different compounds identified so far. belonging to anthocyanidins (more commonly present in form of anthocyanins, their glycoside derivatives), flavones and flavonols (and their glycosides), flavanones, dihydroflavonols such as Dyhydroquercetin (taxifolin), flavan-3-ols, flavan-3,4-diols (leucoanthocyanidins) and to polymeric proanthocyanidins. Flavonoids are especially common in leaves, flowering tissues, and woody parts such as stems, barks and roots. They are important for normal growth, development and defense of plants against infection and injury.

Conifer wood species, especially those from the family of Pinaceae are considered rich sources of flavonoid Dihydroquercetin (taxifolin). which relates to dihydroflavonol subclass of flavonoid family. [3,4,5,6,7,8.9,10] Dihydroflavonols are the central intermediates in flavonoid biosynthesis. The major flavonoids present in knotwood of industrial wood species were determined to belong to dihydroflavonols (also named flavanon-ols). Dihydromyricetin and Dihydrokaempferol were found in knotwood from different Populus spp, along with Dihydroquercetin (taxifolin), where all belong to dihydroflavonols. [11] Dihydroquercetin (taxifolin) has also been determined as the major flavonoid constituent of the hark. Dihydroquercetin (taxifolin) has been detected in knotwood of aspens. [12] Antonova et al. (1980) has studied the organic soluble extractives from the heartwood of larch. The dominant flavonoids found were quercetin (11% of the total amount flavonoids). dihydroquercetin (69%) and dihydrokaempferol. [13] The solvent-soluble constituents of the heartwood of tamarack larch have also been investigated. The flavanonols Dihydroquercetin (taxifolin) and Dihydrokaempferol (aromadendrin) were isolated in 0.30 and 0.05% yield and a trace amount of quercetin was obtained. [14] Bark of shore pines has high contents of the dihydroflavonols Dihydromyricetin and Dihydroquercetin (taxifolin). [15]

Before this invention, it has been known that the extracts of different parts of conifer wood species contain a variety of compounds, one of which is flavonoid Dihydroquercetin (taxifolin). This bioactive substance is available to solvent extraction since Dihydroquercetin (taxifolin) presents in porous structure of wood in form of free molecule which means that molecule is not chemically bonded to the polymers of the cell walls, the structural wood components: cellulose, hemicelluloses and lignins. Dihydroquercetin (taxifolin) is rather associated to these structural polymers through low-energy intermolecular interactions, or simply deposited in the cell lumens or other types of cavities. Being freely positioned in the porous system of lignocellulosic materials Dihydroquercetin (taxifolin) molecule can be solubilized by different solvents according to its physicochemical characteristics. Such substances as Dihydroquercetin (taxifolin) are collectively named the extractives to refer to their common solubility in different solvent systems, but in fact they comprise a vast multitude of chemical structures.

Although it is been mentioned in the literature [3-15] that certain flavonoids, particularly Dihydroquercetin (taxifolin), occurs in wood, including stems, bark and roots, so far, no practically useful unified method has been presented for isolating such compound from these wood parts of conifer wood species.

### SUMMARY OF THE INVENTION

In one general aspect, the invention relates to combining the process of isolation of flavonoid Dihydroquercetin (taxifolin) from wood with the process of utilization of wood in manufacturing various mechanical wood products. The object of this invention is thus to i) provide practically useful sources for this useful flavonoid substance and ii) improve the economy for the manufacturing processes of mechanical wood products in that by-products, hitherto used only for energy production and other non-extraction purposes, are offered a new use as sources for flavonoid Dihydroquercetin (taxifolin).

Thus, this invention relates to a method for isolating of flavonoid Dihydroquercetin (taxifolin) from wood parts including lower portions of the trunk, referred to as butt logs, bark and roots, said method comprising the steps of

a) extracting
i) the particle-reduced chip fraction obtained by chipping or subdividing into chips the butt logs and roots, or
ii) dry cork fraction of the bark or powdered bark obtained as a residue in finishing of mechanical wood products with a polar solvent, and
   b) recovering the extract,
   wherein the wood is a hardwood or a coniferous wood selected from the group consisting of
wood of Larix genus;
spruce wood of the genus Picea;
fir wood of the genus Abies;
pine wood of the genus Pinus;
wood of Pseudotsuga genus and
   wherein the isolated prime substance from all mentioned genus is a flavonoid such as Dihydroquercetin (taxifolin).

The above aspects, advantages and features are of representative embodiments only. It should be understood that they are not to be considered limitations on the invention as defined by the claims. Additional features and advantages of the invention will become apparent in the following description, from the drawings, and from the claims.

### BRIEF DESCRIPTION OF THE DRAWINGS

The invention is illustrated by way of example and not limitation and the figures of the accompanying drawings in which like references denote like or corresponding parts, and in which:

FIG. 1 shows most suitable wood raw material for isolating flavonoid Dihydroquercetin (taxifolin).

FIG. 2 shows schematically a wood material.

FIG. 3 shows a schematic diagram of the system utilized in the process of isolation of flavonoid Dihydroquercetin (taxifolin).

### DETAILED DESCRIPTION OF THE INVENTION

The term “flavonoids” shall be understood to cover anthocyanidins, flavones and flavonols, flavanones, dihydroflavonols, flavan-3-ols, flavan-3,4-diols (leucoanthocyanidins) and polymeric proanthocyanidins.

All these groups are mainly hydrophilic substances that can be extracted with polar, i.e. hydrophilic solvents.

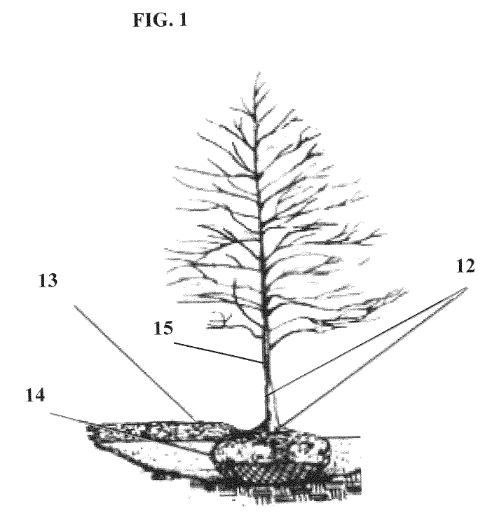

The term “butt logs”, designated with a reference numeral 12 in FIG. 1, shall be understood to include the “logs”, i.e. the lower part of the tree trunk that is stem by maximum 1 meter height from the soil surface 13.

The term “roots” or “root ball”, designated with a reference numeral 14 in FIG. 1, shall be understood to include the “organ of tree”, i.e. the part of the tree that typically lies below the soil surface 13.

The term “bark”, designated with a reference numeral 15 in FIG. 1, shall be understood to include the tissues outside of the vascular cambium, i.e., the outermost layers of stems and roots of woody plants.

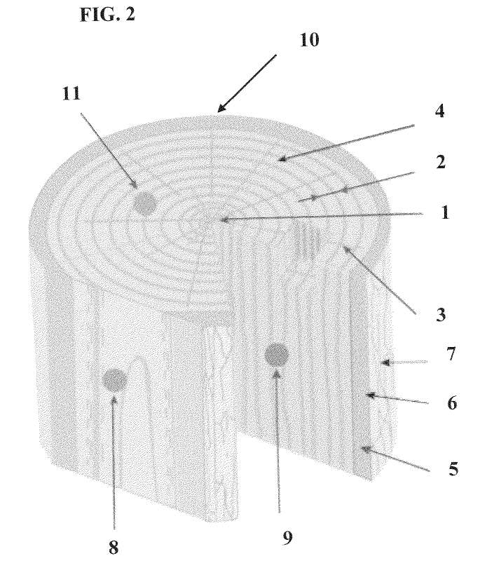

FIG. 2 illustrates different terms used with respect to the wood material 10, where reference numeral 1 designates pith; reference numeral 2 designates the annual ring; reference numeral 3 designates the primary wood ray (pith ray); reference numeral 4 designates the secondary wood ray (from outside in); reference numeral 5 designates cambium; reference numeral 6 designates the inner bark; reference numeral 7 designates the outer bark; section 11 refers to the horizontal section; section 9 refers to the radial section; and section 8 refers to the tangential section. It was determined that the content of extractives of flavonoid nature such as Dihydroquercetin (taxifolin) increases together with density and make a more durable wood, just as a more intensive formation of heartwood makes a more durable tree. The best strength properties and the best durability are found where the growth has been slow, meaning that wider annual rings give lower durability because of a lower basic density. Heartwood from the lower part of the stem will. in general, be more durable than from higher up in the stem, just as the durability increases from the pith outwards. More specifically, the ethanol extractives of flavonoid nature such as Dihydroquercetin (taxifolin) increase between 1.2% and 2.2% per 100 rings increment, which is equivalent to 104 and 175 rings, respectively, to double extractive concentration. Double concentration was reached within a period of well over 200 tree-rings. Flavonoids. which are ethanol-soluble extractives doubled every 10 cm or less of radial distance.

The “particle reduced chip fraction” means the rejected fraction obtained by sizing or grinding to a preferred dimension of the chips aimed for the manufacturing processes of mechanical wood products. This chip fraction comprises, in addition to butt logs and roots, considerable amounts of “normal wood”, i.e., wood usable in the manufacturing processes of mechanical wood products and is used as the material. The wood should be subdivided into chips, preferably having an average length of from about 5 mm to about 20 mm and an average width of from about 3 mm to 10 mm. The particle reduced bark fraction comprises dry cork fraction of the bark or powdered bark usable in manufacturing processes of mechanical wood products.

Although it is possible to use the particle reduced chip fraction and dry cork fraction of the bark or powdered bark as such for extraction of flavonoid substances such as Dihydroquercetin (taxifolin), it may be preferable to first separate the material into a butt log fraction, a root fraction and bark fraction and to use these fractions for each fraction extraction. Both “butt log fraction” and “roots fraction” means the “normal wood” that can be led to the process of obtaining mechanical wood products. “Dry cork fraction of the bark” or “powdered bark” means usable material in the manufacturing processes of mechanical wood products. This separation of fractions can be made directly from the material and can first be refined before subdividing into chips or powdered stage.

Butt logs, roots and bark may be obtained as a waste material or by-product of the logging process, which includes cutting, skidding, on-site processing with utilization of the entire tree including roots, bark, branches and tops. The utilized material can be further chipped or powdered and used for the production of clean electricity or heat.

The “polar solvent” is either a single polar agent, or a mixture of two or more polar agents, where said polar agent or agents have a dielectric constant that is greater than 3, determined at 25° C. As examples of polar solvents, can be mentioned pure water only, and mixtures of water and alcohol, such as water and ethanol.

For extraction, dried wood or raw wood material can be used.

Although the extraction can be physically integrated with the utilization of wood during manufacturing mechanical wood products, the extraction can alternatively be carried out as a separate process, because the butt logs, roots and a bark, especially particle reduced chip fraction of butt logs and roots and dry cork fraction of the bark or powdered bark, can easily be transported and stored for later processing.

The amount of flavonoid substance in butt logs, roots and bark varies greatly and depends on each qualified wood material in question and the wood species used. Therefore, the extract derived from the extraction stage may contain considerable concentrations of flavonoid substance, and may therefore, depending on the purpose, be used as such without further purification for obtaining crude flavonoid yield.

In case further purification is needed, the methods to be used depend, inter alia, on the desired degree of purity of the substance. As examples of useful purification methods, can be mentioned chromatography and\or crystallization.

As important flavonoid substance to be isolated by the method of this invention is Dihydroquercetin (taxifolin). However, the term “flavonoid substance” is not restricted to this compound.

“Dihydroquercetin (taxifolin)” is the compound having molecule structure based on C6-C3-C6 skeleton consisting of two aromatic rings joined by a three carbon link with the absence of the C2-C3 double bond and have two chiral carbon atoms in position 2 and 3. The A ring of the flavonoid structure being acetate derived (3×C2) and the C and B rings originating from cinnamic acid derivatives (phenylpropanoid pathway). Consequently, the B-ring can be either in the (2S)- or (2R)-configuration. The C-3 atom of dihydroflavonol Dihydroquercetin (taxifolin) bears both a hydrogen atom and a hydroxyl group, and is therefore an additional center of asymmetry. Thus, four stereoisomers are possible for each dihydroflavonol structure, (2R,3R), (2R,3S), (2S,3R), and (2S,3S). All four configurations have been found in naturally occurring dihydroflavonols, but the (2R,3R)-configuration is by far the most common.

The isolation of flavonoid substances or Dihydroquercetin (taxifolin) from butt logs, roots and bark is very advantageous compared to the utilization of other sources. In these “butt log fraction”, “roots fraction” and “dry cork fraction of the bark or powdered bark” sources, the concentration of flavonoid substances is 2 to 20 times higher than in normal wood. Many of these compounds cannot be located in normal wood. As a result, about 20-70% of the extract obtained according to this method, may be the flavonoid agent or agents. Another feature is that a certain compound may be present as the dominating compound of the derived flavonoid group of substances. For example, Dihydroquercetin (taxifolin) may be about 65-85% of the flavonoids derived from larch wood butt logs.

This invention thus offers a unique method for deriving the desired flavonoid compound or Dihydroquercetin (taxifolin) in high concentrations in the extract. Along with this advantage, the wood material utilized for the extraction is material that hitherto has been regarded as a wood fraction useful as energy source only.

### Method of Isolation

For the purpose of illustration, the isolation of flavonoid Dihydroquercetin (taxifolin) from wood of the Larch tree is described herein by way of example, however the methods can be readily be adapted for the isolation of compound from other fibrous plant materials such as other types of conifer trees. Prior to processing, the fibrous plant material, such as wood, roots or bark, for example derived from a tree of the Larix genus, optionally may be sized to a preferred dimension using methods available in the art. In a typical system, the wood material butt logs 12, roots 14 and bark 15 must be cut and ground or processed by a machine known in the art to comminute raw wood material into wood particles. The preferred size of the Larch wood particles depends upon the type of equipment used to process the wood particles. The raw wood particles are then fed to an inlet hopper for storage until the wood particles are required for the next step.

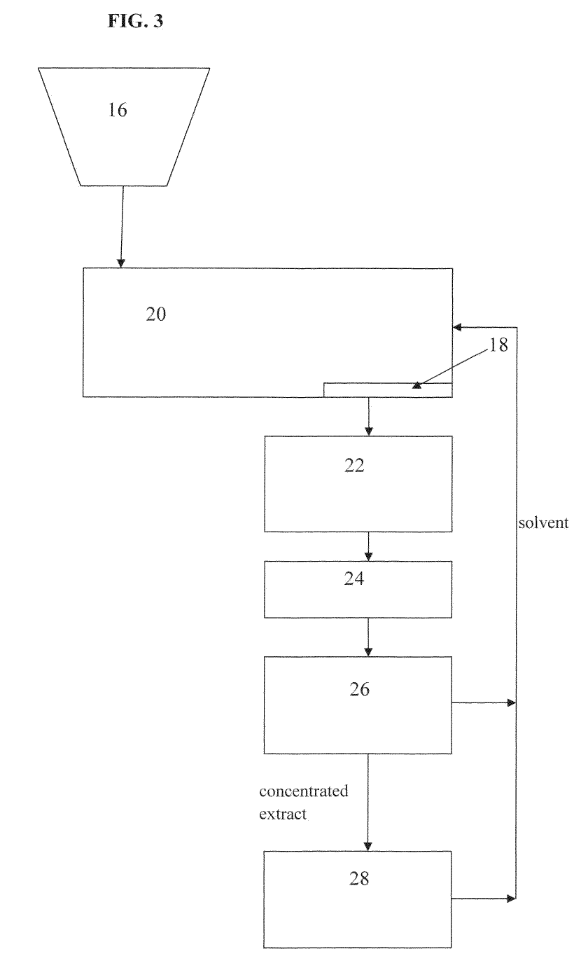

As shown in FIG. 3, the properly sized and ground prepared wood particles are ready to be processed, they are transported from the inlet hopper via a chute 16 to an extractor unit 20. Extractor unit is well known in the art, and typically include an inlet and an outlet, level detectors for level control, and a solvent pretreatment inlet for optionally mixing prepared solvent mixture with the wood particles. The solvent mixture pretreatment is used to thaw or soften the wood particles so that the energy required to obtain exudate from the wood particles in later processing steps is decreased.

Method for deriving the desired flavonoid compound or Dihydroquercetin (taxifolin) prefers that extraction is performed in a vacuum system where energy is used to heat the solvent mixture, a mixture of two or more polar agents, in contact with wood particles in order to extract flavonoid Dihydroquercetin (taxifolin) from the wood particles. The extraction conditions including solid/liquid ratio, extraction time, extraction temperature and degree of vacuum to be optimized. Optimized conditions considered to be with solid/liquid ratio 1:4 and 1:6, extraction at temperature of 38° C.-40° C., and using ethanol as a food grade solvent in mixture from 20 to 40% of ethanol with 80 to 60% of water (v/v) ethanol/water ratio.

The boiling point of the extraction solvent mixture in vacuum is lower than that at normal air pressure. Thus, the extraction is performed at a lower temperature from 25° C. to maximum 40° C. It is good for preventing the degradation of thereto sensitive compound Dihydroquercetin (taxifolin). Also the solvent mixture can be kept boiling and refluxing at lower temperatures such as in the optimum range of 30° C.-38° C. It is preferred for mixing wood material with solvent mixture and extracting compound out of the wood material. Furthermore, the air in the extraction system is mostly pumped out, so the oxidation of the thermosensitive compound is avoided or reduced since there is little oxygen in the process of extraction.

The degree of vacuum and extraction temperature had obvious effect on the extraction yields of flavonoid Dihydroquercetin (taxifolin). The degree of vacuum can be adjusted following the extraction temperature at 30° C.-38° C. to cause solvent mixture to have higher capacity at such temperatures, and sufficient enough to cause the solvent to reflux continuously. Moreover, the mixing of solvent and wood particle at pre-extraction stage leads to the higher extraction yields of the compound.

The principle of solid-liquid extraction in a vacuum system is that when a solid material comes in contact with a solvent mixture, the soluble in mixture components in the solid wood particles, such as flavonoid Dihydroquercetin (taxifolin), move to the solvent. Thus, solvent extraction wider vacuum of wood material results in the mass transfer of soluble active principle (Dihydroquercetin or taxifolin) to the solvent, and this takes place in a concentration gradient. Since mass transfer of the active principle Dihydroquercetin (taxifolin) also depends on its solubility in the solvent mixture, heating the solvent mixture can enhance the mass transfer.

For extraction, the solvent or solvent mixture is chosen as a function of the type of flavonoid Dihydroquercetin (taxifolin) required. Polarity is an important consideration here. More polar flavonoid Dihydroquercetin (taxifolin) is extracted with ethanol or ethanol/water mixtures. The bulk of extractions of flavonoid-containing material are still performed by simple direct solvent extraction in a vacuum system.

The obtained water-ethanol exudate or extract often contains numerous substances such as sugars and resin, fats, terpenes, lignans, etc. Consequently, a purification step is necessary. The extract so obtained is separated out from the marc (exhausted wood material) by allowing it to trickle into a holding tank 22 through the built-in false bottom of the extractor, which is covered with a filter cloth 18. The marc is retained at the false bottom, and the extract is received in the holding tank. From the holding tank, the extract is pumped into a sparkler filter 24 to remove fine or colloidal particles from the extract.

The enriched extract from extractor, known as miscella, is fed into a wiped film vacuum evaporator 26 where it is concentrated under vacuum to produce a thick concentrated extract. The concentrated extract is further fed into a vacuum chamber dryer 28 to produce a solid paste mass free from solvent. The solvent recovered from the wiped film evaporator 26 and vacuum chamber dryer 28 is recycled back to the extractor for the next batch of wood material. The concentrated extract thus obtained is used directly for the further processed for isolation of flavonoid Dihydroquercetin (taxifolin).

The state of art is cautious vacuum evaporation, in which evaporation temperatures do not exceed 45° C. The temperature in relation to the evaporation time is of special importance for quality of this step, especially if the extract contains thermolabile constituents like flavonoid Dihydroquercetin (taxifolin).

The aqueous concentrated extract is then concentrated in two stages by freezing, using Gulf crystallisers, for example. After the first stage, the extract is in the form of a pumpable slurry which is centrifuged to provide a concentrated liquor containing about 40% total solids, which is further concentrated to about 50% total solids in the second stage. The Dihydroquercetin (taxifolin) extract is then frozen as described below. The frozen product is broken up and ground to a particle size of 0.1 mm to 0.2 mm. After freeze-drying, the powder is agglomerated in a suitable agglomeration chamber. The agglomerated powder, having a density of tip to 0.5 g/cm³, may be packed in an air-tight containers in an inert atmosphere.

Drying of the concentrated extract should preferably be carried out under controlled conditions such that the finished product possesses an appropriate density and colour. Paste mass extract is frozen to a solid mass in the form of plates or lumps. At the first stage, paste mass of extract may be cooled to a temperature of −12° C. to −29° C. while at the second stage, cooling may be conducted at a lower temperature, for example, between −40° C. and −70° C. In general, it is desirable for the residence time of the paste mass extract in the cooling volume to be not less than about 7 minutes, and may be as long as 25 minutes.

At the end of the freeze drying the extract is removed as a continuous rigid sheet which may then be broken up into fragments suitable for grinding. These fragments may, for example, be ground to a particle size which is preferably within the range. The ground particles are then freeze-dried in conventional cabinets, on trays which are loaded to a layer thickness of, for example up to 0.1 mm. The sublimation of the ice crystals is effected under a high vacuum, of about 150 to 175 microns, and generally lasts around 7 hours. The agglomerated powder, having a density of 0.2 to 0.5 g/cm³, may be packed in air-tight containers in an inert atmosphere or packed as desired.

Different purity extracts of natural products which show desirable biological activity are subjected to activity-guided fractionation until an active component is isolated and identified. This exploratory process of fractionation typically involves suboptimal chromatographic conditions; hence, in order to avoid destruction of potentially labile component Dihydroquercetin (taxifolin), utmost care must be taken throughout the entire process of isolation.

High-Performance Liquid Chromatography (HPLC) is becoming by far the most popular technique for the separation of flavonoids, both on preparative and analytical scales. The difference between the analytical and preparative methodologies is that analytical HPLC does not rely on the recovery of a sample, while preparative HPLC is a purification process and aims at the isolation of a pure substance from a mixture. Process-scale HPLC is the choice for isolating valuable molecules such as Dihydroquercetin (taxifolin) with desired purity for commercialization. With the increasing demand for flavonoid products, nutraceuticals, and natural products for health care all over the world, flavonoids extract manufacturers have started using the most appropriate extraction technologies in order to produce extracts of defined quality with the least variations from batch to batch.

In the preceding specification, the invention has been described with reference to specific exemplary embodiments thereof. It will however, be evident that various modifications and changes may be made thereto without departing from the broader spirit and scope of the invention as set forth in the claims that follow. The specification and drawings are accordingly to be regarded in an illustrative manner rather than a restrictive sense.

1. A method for isolating a flavonoid from wood including butt logs, bark and roots, said method comprising the steps of:
a) forming a particle-reduced chip fraction by subdividing said butt logs and said roots into chips having an average length of 5 mm to 20 mm and a width of 3 mm to 10 mm;
vacuum extracting an extract from a combination of a polar solvent with the particle-reduced chip fraction, and a dry cork fraction of the bark obtained as a residue in finishing of mechanical wood products, the step of vacuum extracting being performed in a vacuum system by heating said combination to a temperature of 25° C. to 40° C.;
c) recovering said extract obtained as a result of said step of extracting; and
d) freeze-drying the recovered extract,
wherein the wood is a coniferous wood selected from the group consisting of a wood of Larix genus, a spruce wood of the genus Picea, a fir wood of the genus Abies, a pine wood of the genus Pinus, and a wood of Pseudotsuga genus, and wherein an isolated prime substance from all mentioned genus is a flavonoid and the wood is a hardwood.

2. The method according to claim 1, wherein the polar solvent a single polar agent, or a mixture of two or more polar agents.

3. The method according to claim 2, wherein said two polar agents in said mixture further comprise from 20% to 40% of ethanol and 80% to 60% of water (v/v).

4. The method according to claim 1, wherein said step of vacuum extracting is performed in the vacuum system in which an energy is used to heat said combination of said polar solvent in contact with said wood particles in order to extract said flavonoid Dihydroquercetin (taxifolin) from said wood particles.

5. The method according to claim 3, wherein said step of vacuum extracting is performed in the vacuum system in which an energy is used to heat said mixture in contact with said wood particles in order to extract said flavonoid from said wood particles.

6. The method according to claim 1, wherein said extract is further purified for releasing a desired flavonoid substance.

7. The method according to claim 6, wherein said desired flavonoid substance is Dihydroquercetin (taxifolin).

8. The method according to claim 6, further comprising freeze-drying the purified extract.

9. The method according to claim 1, wherein the hardwood is larch and the isolated flavonoid is Dihydroquercetin (taxifolin).
